SEQUENCE LISTING (12 sequences)

SEQ ID NO: 1 - protein, 376 aa, Homo sapiens
KSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 238 aa, Homo sapiens
VSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 3 - protein, 441 aa, Homo sapiens
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 4 - protein, 576 aa, Homo sapiens
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 5 - protein, 424 aa, Homo sapiens
HPDRIIFPNHACEDPPAVLLEVQGTLQRPLVRDSRTSPANCTWLILGSKEQTVTIRFQKLHLACGSERLTLRSPLQPLISLCEAPPSPLQLPGGNVTITYSYAGARAPMGQGFLLSYSQDWLMCLQEEFQCLNHRCVSAVQRCDGVDACGDGSDEAGCSSDPFPGLTPRPVPSLPCNVTLEDFYGVFSSPGYTHLASVSHPQSCHWLLDPHDGRRLAVRFTALDLGFGDAVHVYDGPGPPESSRLLRSLTHFSNGKAVTVETLSGQAVVSYHTVAWSNGRGFNATYHVRGYCLPWDRPCGLGSGLGAGEGLGERCYSEAQRCDGSWDCADGTDEEDCPGCPPGHFPCGAAGTSGATACYLPADRCNYQTFCADGADERRCRHCQPGNFRCRDEKCVYETWVCDGQPDCADGSDEWDCSYVLPRK

SEQ ID NO: 6 - protein, 121 aa, Homo sapiens
WSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 7 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 8 - protein, 250 aa, Homo sapiens [sequence omitted]

SEQ ID NO: 9 - protein, 238 aa, Homo sapiens
QVSATASPSGSLGAPDCPEVCTCVPGGLASCSALSLPAVPPGLSLRLRALLLDHNRVRALPPGAFAGAGALQRLDLRENGLHSVHVRAFWGLGALQLLDLSANQLEALAPGTFAPLRALRNLSLAGNRLARLEPAALGALPLLRSLSLQDNELAALAPGLLGRLPALDALHLRGNPWGCGCALRPLCAWLRRHPLPASEAETVLCVWPGRLTLSPLTAFSDAAFSHCAQPLALRDLAV

SEQ ID NO: 10 - protein, 118 aa, Homo sapiens
LRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 11 - protein, 145 aa, Homo sapiens
APKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 12 - protein, 636 aa, Homo sapiens
EEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

(19) United States
(12) Patent Application Publication
(10) Pub. No.: US 2021/0080462 A1
(43) Pub. Date: Mar. 18, 2021

(54) Reagents and Methods for Monitoring Breast Cancer Therapy

(71) Applicant: SANFORD HEALTH, Sioux Falls, SD (US)

(72) Inventors: Kristi A. EGLAND, Sioux Falls, SD (US); Rick L. EVANS, Sioux Falls, SD (US); James V. POTTALA, Sioux Falls, SD (US)

(21) Appl. No.: 17/103,318

(22) Filed: Nov. 24, 2020

Related U.S. Application Data
(63) Continuation of application No. 15/781,373, filed on Jun. 4, 2018.
(60) Provisional application No. 62/266,189, filed on Dec. 11, 2015.

(51) Int. Cl. G01N 33/574

(52) U.S. Cl. CPC G01N 33/57415; G01N 2800/60; G01N 2800/54; G01N 2800/52

(57) ABSTRACT

Disclosed herein are compositions including reagents for detecting human autoantibodies against at least two proteins selected from the group consisting of A1AT, ANGPTL4, LRP10, GFRA1, LGALS3, CST2, DKK1, CAPC, GRP78, and GRN, wherein at least one of the antibody detection markers is selected from the group consisting of A1AT, LGALS3, and CAPC, and their use in monitoring efficacy of breast cancer therapy and breast cancer recurrence.

### CROSS REFERENCE

This application claims priority to U.S. Provisional Patent Application Ser. No. 62/266,189 filed Dec. 11, 2015, incorporated by reference herein in its entirety.

### FEDERAL FUNDING STATEMENT

This invention was made with government support under Grant No. P20GM103548 awarded by the National Institutes of Health. The government has certain rights in the invention.

### BACKGROUND

The 5-year survival rate for women diagnosed with local breast cancer (BCa) is 98.6%. Survival declines to 83.8% for regional stage and plummets to 23.3% for distant stage [3]. Only 5% of U.S. women present with metastatic BCa when first diagnosed [4]; however, most BCa-related deaths are due to incurable metastatic disease [5], not the primary diagnosis. Unfortunately, recurrence of BCa is most often found when patients report symptoms, such as shortness of breath, chronic cough, weight loss or bone pain.

### SUMMARY OF THE INVENTION

In one aspect, the invention provides compositions comprising at least 2 antibody detection markers, wherein the antibody detection markers comprise reagents for detecting human autoantibodies against at least two proteins selected from the group consisting of A1AT (Alpha-1 antitrypsin) (SEQ ID NO:7), ANGPTL4 (Angiopoietin-like 4) (SEQ ID NO:1), LRP10 (LDL Receptor Related Protein 10) (SEQ ID NO:5), GFRA1, (GDNF Family Receptor Alpha 1) (SEQ ID NO:3), LGALS3 (Galectin-3) (SEQ ID NO:8), CST2 (Cystatin SA) (SEQ ID NO:6), DKK1 (Dickkopf WNT Signaling Pathway Inhibitor 1) (SEQ ID NO:2), CAPC (Cytokeratin-Associated Protein in Cancer) (SEQ ID NO:9), GRP78 (78 kDa glucose-regulated protein) (SEQ ID NO: 12) and GRN (Granulin) (SEQ ID NO:4), wherein at least one of the proteins is selected from the group consisting of A1AT, LGALS3, and CAPC. In one embodiment, the composition includes reagents for detecting human autoantibodies against at least 3 proteins selected from the group consisting of A1AT, ANGPTL4, LRP10, GFRA1, LGALS3, CST2, DKK1, CAPC, GRP78, and GRN. In another embodiment, the composition includes reagents for detecting human autoantibodies against at least 5, 6, 7, 8, or all 9 proteins in the recited group. In further embodiments, the composition consists of between 2 and 1000 antibody detection markers, or between 4 and 500 antibody detection markers. In another embodiment, the composition includes reagents for detecting human autoantibodies against at least 2, 3, 4, or all 5 of ANGPTL4, GFRA1, LGALS3, DKK1 and GRN. In a further embodiment, the composition further comprises reagents for detecting human autoantibodies against one or both of GAL1 and MUC1. In one embodiment, the reagents for detecting human autoantibodies comprise the at least two proteins, or antigenic fragments thereof. In another embodiment, the at least two proteins, or antigenic fragments thereof comprise native extracellular domains and/or native secreted proteins or antigenic fragments thereof. In a further embodiment, the reagents are detectably labeled. In a still further embodiment, the at least two proteins, or antigenic fragments thereof, are expressed as a fusion with a detectable domain, including but not limited to an Fc domain. In another embodiment, the reagents are immobilized on a surface of a solid support.

In another aspect, the invention provides methods for monitoring breast cancer therapy, comprising
(a) contacting a bodily fluid sample from a subject who is undergoing or has undergone breast cancer therapy with one or more reagents for detecting autoantibodies against one or more proteins selected from the group consisting of A1AT, ANGPTL4, LRP10, GFRA1, LGALS3, CST2, DKK1, CAPC, GRP78, and GRN; and
(b) determining an amount of autoantibodies against the one or more proteins in the bodily fluid sample;
wherein a decrease in the amount of autoantibodies relative to a control, such as a baseline level of autoantibodies in a similar bodily fluid sample from the subject indicates efficacy of the breast cancer therapy in the subject.

In another aspect, the invention provides methods for prognosing breast cancer recurrence, comprising
(a) contacting a bodily fluid sample from a subject who has received breast cancer therapy with one or more reagents for detecting autoantibodies against one or more proteins selected from the group consisting of A1AT, ANGPTL4, LRP10, GFRA1, LGALS3, CST2, DKK1, CAPC, GRP78, and GRN; and
(b) determining an amount of autoantibodies against the one or more proteins in the bodily fluid sample;
wherein an increase in the amount of autoantibodies relative to a baseline level of autoantibodies in a control, such as a similar bodily fluid sample from the subject indicates a likelihood of breast cancer recurrence in the subject.

In various embodiments, the reagents comprise reagents for detecting autoantibodies 3, 4, 5, 6, 7, 8, or all 9 of the recited proteins. In another embodiment, the reagents comprise the composition of any embodiment or combination of embodiments of the invention.

In one embodiment, the one or more reagents comprise 2, 3, 4, or all 5 proteins, or antigenic fragments thereof, selected from the group consisting of ANGPTL4, GFRA1, LGALS3, DKK1 and GRN.

In another aspect, the invention provides methods for monitoring breast cancer therapy, comprising
(a) contacting a bodily fluid sample from a subject who is undergoing or has undergone breast cancer therapy with one or more reagents for detecting autoantibodies against GRP78; and
(b) determining an amount of autoantibodies against GRP78 in the bodily fluid sample;
wherein an increase in the amount of autoantibodies against GRP78 relative to a control, such as a baseline level of autoantibodies in a similar bodily fluid sample from the subject indicates efficacy of the breast cancer therapy in the subject.

In a further aspect, the invention provides methods for prognosing breast cancer recurrence, comprising
(a) contacting a bodily fluid sample from a subject who has received breast cancer therapy with one or more reagents for detecting autoantibodies against GRP78; and
(b) determining an amount of autoantibodies against GRP78 in the bodily fluid sample;
wherein a decrease in the amount of autoantibodies relative to a baseline level of autoantibodies against GRP78 in a control, such as a similar bodily fluid sample from the subject indicates a likelihood of breast cancer recurrence in the subject. In another embodiment, the contacting comprises use of ELISA. In a further embodiment, the bodily fluid sample comprises a serum sample from the subject. In one embodiment, the subject has had surgery to remove the primary tumor prior to carrying out the method of the invention. In another embodiment, the subject is receiving or has received radiation therapy and chemotherapy, or hormonal therapy and chemotherapy. In a further embodiment, the contacting comprises use of Longitudinal Assay Screening, wherein all target biomarkers may be detected and quantitated within a single test and dilution. In a still further embodiment, the bodily fluid sample comprises a blood sample from the subject. In one embodiment, if no decrease is determined in the amount of autoantibodies relative to a baseline level of autoantibodies in a similar bodily fluid sample from the subject, the method further comprises altering the breast cancer therapy being administered to the subject. In another embodiment, the method is used to detect breast cancer recurrence.

### BRIEF DESCRIPTION OF THE FIGURES

FIG. 1. Observed geometric mean changes (with 95% confidence intervals) from baseline for autoantibodies levels against 11 tumor-associated antigens according to treatment regimen after 12 months follow-up. *indicates p-value<0.05. There were no significant changes observed for surgery only or individual therapies (i.e. hormonal, radiation or chemotherapy).

FIG. 2. Antibody response against ERBB2-rFc in patients diagnosed with HER2 positive breast cancer treated with HERCEPTIN®. Three longitudinal blood draws were collected, immediately before surgery, 6 months and 12 months after surgery. The solid lines indicate patients who were still receiving HERCEPTIN® therapy at their 12-month visit, and the dashed lines represent patients that discontinued HERCEPTIN® therapy prior to their 12-month visit. A light, green circle represents patient BC-166 because only the baseline blood draw was acquired. Geometric mean values were 48, 1206 and 600 over time. Follow up levels were significantly greater than baseline (p<0.0001), but 6 and 12-month levels were not different (p=0.09).

FIG. 3. Observed geometric mean changes (with 95% confidence intervals) from baseline for autoantibodies levels against 11 tumor-associated antigens according to treatment regimen after 6 months follow-up. * indicates p-value<0.05. There were no significant changes observed for surgery only or individual therapies (i.e. hormonal, radiation, or chemotherapy).

### DETAILED DESCRIPTION OF THE INVENTION

All references cited are herein incorporated by reference in their entirety.

Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{rd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

In a first aspect, the present invention provides compositions comprising at least 2 antibody detection markers, wherein the antibody detection markers comprise reagents for detecting human autoantibodies against at least two proteins selected from the group consisting of A1AT (Alpha-1 antitrypsin) (SEQ ID NO:7), ANGPTL4 (Angiopoietin-like 4) (SEQ ID NO:1), LRP10 (LDL Receptor Related Protein 10) (SEQ ID NO:5), GFRA1, (GDNF Family Receptor Alpha 1) (SEQ ID NO:3), LGALS3 (Galectin-3) (SEQ ID NO:8), CST2 (Cystatin SA) (SEQ ID NO:6), DKK1 (Dickkopf WNT Signaling Pathway Inhibitor 1) (SEQ ID NO:2), CAPC (Cytokeratin-Associated Protein In Cancer) (SEQ ID NO:9), GRP78 (78 kDa glucose-regulated protein) (SEQ ID NO: 12) and GRN (Granulin) (SEQ ID NO:4), Wherein at least one of the proteins is selected from the group consisting of A1AT, LGALS3, and CAPC.

The inventors have unexpectedly discovered that autoantibodies against the recited proteins provide an indication of efficacy of therapy for a subject being treated for BCa, and/or BCa recurrence. Thus, the compositions of the invention can be used, for example, in diagnostic assays to monitor efficacy of breast cancer therapy, and/or recurrence. In one embodiment, the composition includes reagents for detecting human autoantibodies against at least two proteins selected from the group consisting of A1AT, ANGPTL4, LRP10, GFRA1, LGALS3, CST2, DKK1, CAPC, GRP78, and GRN.

In various embodiments, the composition comprises or consists of reagents for detecting human autoantibodies against at least three, four, five, six, seven, eight, or all nine proteins in the recited group. In various further embodiments, the composition comprises or consists of reagents for detecting human autoantibodies against 2, 3, 4, or all 5 of ANGPTL4, GFRA1, LGALS3, DKK1 and GRN.

In a further embodiment, the composition further comprises reagents for detecting human autoantibodies against one or both of GAL1 and MUC1.

In various further embodiments, the composition comprises or consists of between 2-1000, 3-1000, 4-1000, 5-1000, 6-1000, 7-1000, 8-1000, 9-1000, 2-500, 3-500, 4-500, 5-500, 6-500, 7-500, 8-500, 9-500, 2-100, 3-100, 4-100, 5-100, 6-100, 7-100, 8-100, 9-100, 2-50, 3-50, 4-50, 5-50, 6-50, 7-50, 8-50, 9-50, 2-25, 3-25, 4-25, 5-25, 6-25, 7-25, 8-25, or 9-25 antibody detection reagents.

As will be understood by those of skill in the art, the compositions may include additional antibody detection markers and controls as is appropriate for an intended use of the composition.

The antibody detection markers may be any suitable reagents that can be used to detect antibodies against the recited proteins, including but not limited to the recited protein, a secreted version of the protein (such as a native secreted form of the protein), or an extracellular domain of the protein. Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular protein. Thus, in one embodiment the antibody detection marker can be a secreted or membrane protein of the recited protein. Exemplary amino acid sequences of the recited human proteins are shown below, as noted:
Full length protein without signal sequence: ANGPTL4, A1AT, CST2, DKK1, GFRA1, GRN, GRP78, GALL MUC1.
Extracellular domain region without signal sequence: CAPC, LRP10
Full length (no signal sequence predicted in protein): LGALS3

ANGTPL4
(SEQ ID NO: 1)
KSPRFASWDEMNVLAHGLLQLGQGLREHAERTRSQLSALERRLSACG
SACQGTEGSTDLPLAPESRVDPEVLHSLQTQLKAQNSRIQQLFHKVA
QQQRHLEKQHLRIQHLQSQFGLLDHKHLDHEVAKPARRKRLPEMAQP
VDPAHNVSRLHRLPRDCQELFQVGERQSGLFEIQPQGSPPFLVNCKM
TSDGGWTVIQRRHDGSVDFNRPWEAYKAGFGDPHGEFWLGLEKVHSI
TGDRNSRLAVQLRDWDGNAELLQFSVHLGGEDTAYSLQLTAPVAGQL
GATTVPPSGLSVPFSTWDQDHDLRRDKNCAKSLSGGWWFGTCSHSNL
NGQYFRSIPQQRQKLKKGIFWKTWRGRYYPLQATTMLIQPMAAEAAS
DKK1
(SEQ ID NO: 2)
VSATLNSVLNSNAIKNLPPPLGGAAGHPGSAVSAAPGILYPGGNKYQ
TIDNYQPYPCAEDEECGTDEYCASPTRGGDAGVQICLACRKRRKRCM
RHAMCCPGNYCKNGICVSSDQNHFRGEIEETITESFGNDHSTLDGYS
RRTTLSSKMYHTKGQEGSVCLRSSDCASGLCCARHFWSKICKPVLKE
GQVCTKHRRKGSHGLEIFQRCYCGEGLSCRIQKDHHQASNSSRLHTC
QRH
GFRA1
(SEQ ID NO: 3)
DRLDCVKASDQCLKEQSCSTKYRTLRQCVAGKETNFSLASGLEAKDE
CRSAMEALKQKSLYNCRCKRGMKKEKNCLRIYWSMYQSLQGNDLLED
SPYEPVNSRLSDIFRVVPFISDVFQQVEHIPKGNNCLDAAKACNLDD
ICKKYRSAYITPCTTSVSNDVCNRRKCHKALRQFFDKVPAKHSYGML
FCSCRDIACTERRRQTIVPVCSYEEREKPNCLNLQDSCKTNYICRSR
LADFFTNCQPESRSVSSCLKENYADCLLAYSGLIGTVMTPNYIDSSS
LSVAPWCDCSNSGNDLEECLKFLNFFKDNTCLKNAIQAFGNGSDVTV
WQPAFPVQTTTATTTTALRVKNKPLGPAGSENEIPTHVLPPCANLQA
QKLKSNVSGNTHLCISNGNYEKEGLGASSHITTKSMAAPPSCGLSPL
LVLVVTALSTLLSLTETS
GRN
(SEQ ID NO: 4)
TRCPDGQFCPVACCLDPGGASYSCCRPLLDKWPTTLSRHLGGPCQVD
AHCSAGHSCIFTVSGTSSCCPFPEAVACGDGHHCCPRGFHCSADGRS
CFQRSGNNSVGAIQCPDSQFECPDFSTCCVMVDGSWGCCPMPQASCC
EDRVHCCPHGAFCDLVHTRCITPTGTHPLAKKLPAQRTNRAVALSSS
VMCPDARSRCPDGSTCCELPSGKYGCCPMPNATCCSDHLHCCPQDTV
CDLIQSKCLSKENATTDLLTKLPAHTVGDVKCDMEVSCPDGYTCCRL
QSGAWGCCPFTQAVCCEDHIHCCPAGFTCDTQKGTCEQGPHQVPWME
KAPAHLSLPDPQALKRDVPCDNVSSCPSSDTCCQLTSGEWGCCPIPE
AVCCSDHQHCCPQGYTCVAEGQCQRGSEIVAGLEKMPARRASLSHPR
DIGCDQHTSCPVGQTCCPSLGGSWACCQLPHAVCCEDRQHCCPAGYT
CNVKARSCEKEVVSAQPATFLARSPHVGVKDVECGEGHFCHDNQTCC
RDNRQGWACCPYRQGVCCADRRHCCPAGFRCAARGTKCLRREAPRWD
APLRDPALRQLL
LRP10
(SEQ ID NO: 5)
HPDRIIFPNHACEDPPAVLLEVQGTLQRPLVRDSRTSPANCTWLILG
SKEQTVTIRFQKLHLACGSERLTLRSPLQPLISLCEAPPSPLQLPGG
NVTITYSYAGARAPMGQGFLLSYSQDWLMCLQEEFQCLNHRCVSAVQ
RCDGVDACGDGSDEAGCSSDPFPGLTPRPVPSLPCNVTLEDFYGVFS
SPGYTHLASVSHPQSCHWLLDPHDGRRLAVRFTALDLGFGDAVHVYD
GPGPPESSRLLRSLTHFSNGKAVTVETLSGQAVVSYHTVAWSNGRGF
NATYHVRGYCLPWDRPCGLGSGLGAGEGLGERCYSEAQRCDGSWDCA
DGTDEEDCPGCPPGHFPCGAAGTSGATACYLPADRCNYQTFCADGAD
ERRCRHCQPGNFRCRDEKCVYETWVCDGQPDCADGSDEWDCSYVLPR
K
CST2
(SEQ ID NO: 6)
WSPQEEDRIIEGGIYDADLNDERVQRALHFVISEYNKATEDEYYRRL
LRVLRAREQIVGGVNYFFDIEVGRTICTKSQPNLDTCAFHEQPELQK
KQLCSFQIYEVPWEDRMSLVNSRCQEA
A1AT
(SEQ ID NO: 7)
EDPQGDAAQKTDTSHHDQDHPTFNKITPNLAEFAFSLYRQLAHQSNS
TNIFFSPVSIATAFAMLSLGTKADTHDEILEGLNFNLTEIPEAQIHE
GFQELLRTLNQPDSQLQLTTGNGLFLSEGLKLVDKFLEDVKKLYHSE
AFTVNFGDTEEAKKQINDYVEKGTQGKIVDLVKELDRDTVFALVNYI
FFKGKWERPFEVKDTEEEDFHVDQVTTVKVPMMKRLGMFNIQHCKKL
SSWVLLMKYLGNATAIFFLPDEGKLQHLENELTHDIITKFLENEDRR
SASLHLPKLSITGTYDLKSVLGQLGITKVFSNGADLSGVTEEAPLKL
SKAVHKAVLTIDEKGTEAAGAMFLEAIPMSIPPEVKFNKPFVFLMIE
QNTKSPLFMGKAVVNPTQK
LGALS3
(SEQ ID NO: 8)
MADNFSLHDALSGSGNPNPQGWPGAWGNQPAGAGGYPGASYPGAYPG
QAPPGAYPGQAPPGAYPGAPGAYPGAPAPGVYPGPPSGPGAYPSSGQ
PSATGAYPATGPYGAPAGPLIVPYNLPLPGGVVPRMLITILGTVKPN
ANRIALDFQRGNDVAFHFNPRFNENNRRVIVCNTKLDNNWGREERQS
VFPFESGKPFKIQVLVEPDHFKVAVNDAHLLQYNHRVKKLNEISKLG
ISGDIDLTSASYTMI
CAPC
(SEQ ID NO: 9)
QVSATASPSGSLGAPDCPEVCTCVPGGLASCSALSLPAVPPGLSLRL
RALLLDHNRVRALPPGAFAGAGALQRLDLRENGLHSVHVRAFWGLGA
LQLLDLSANQLEALAPGTFAPLRALRNLSLAGNRLARLEPAALGALP
LLRSLSLQDNELAALAPGLLGRLPALDALHLRGNPWGCGCALRPLCA
WLRRHPLPASEAETVLCVWPGRLTLSPLTAFSDAAFSHCAQPLALRD
LAV
GAL1
(SEQ ID NO: 10)
LRVRGEVAPDAKSFVLNLGKDSNNLCLHFNPRFNAHGDANTIVCNSK
DGGAWGTEQREAVFPFQPGSVAEVCITFDQANLTVKLPDGYEFKFPN
RLNLEAINYMAADGDFKIKCVAFD
MUC1
(SEQ ID NO: 11)
APKPATVVTGSGHASSTPGGEKETSATQRSSVPSSTEKNAFNSSLED
PSTDYYQELQRDISEMFLQIYKQGGFLGLSNIKFRPGSVVVQLTLAF
REGTINVHDVETQFNQYKTEAASRYNLTISDVSVSDVPFPFSAQSGA
GVPG
GRP78
(SEQ ID NO: 12)
EEEDKKEDVGTVVGIDLGTTYSCVGVFKNGRVEIIANDQGNRITPSY
VAFTPEGERLIGDAAKNQLTSNPENTVFDAKRLIGRTWNDPSVQQDI
KFLPFKVVEKKTKPYIQVDIGGGQTKTFAPEEISAMVLTKMKETAEA
YLGKKVTHAVVTVPAYFNDAQRQATKDAGTIAGLNVMRIINEPTAAA
IAYGLDKREGEKNILVFDLGGGTFDVSLLTIDNGVFEVVATNGDTHL
GGEDFDQRVMEHFIKLYKKKTGKDVRKDNRAVQKLRREVEKAKRALS
SQHQARIEIESFYEGEDFSETLTRAKFEELNMDLFRSTMKPVQKVLE
DSDLKKSDIDEIVLVGGSTRIPKIQQLVKEFFNGKEPSRGINPDEAV
AYGAAVQAGVLSGDQDTGDLVLLDVCPLTLGIETVGGVMTKLIPRNT
VVPTKKSQIFSTASDNQPTVTIKVYEGERPLTKDNHLLGTFDLTGIP
PAPRGVPQIEVTFEIDVNGILRVTAEDKGTGNKNKITITNDQNRLTP
EEIERMVNDAEKFAEEDKKLKERIDTRNELESYAYSLKNQIGDKEKL
GGKLSSEDKETMEKAVEEKIEWLESHQDADIEDFKAKKKELEEIVQP
IISKLYGSAGPPPTGEEDTAEKDEL

In a further embodiment, the antibody detection marker is a protein, such as those disclosed above, that is in its native form. As disclosed in the accompanying examples, the inventors utilized a eukaryotic expression system to generate conformation-carrying tumor antigens that are properly folded and contain non-continuous epitopes for use in the detection of autoantibodies. In another embodiment, the reagents comprise the at least two proteins, or antigenic fragments thereof, and wherein the at least two proteins, or antigenic fragments thereof, are expressed as a fusion with a detectable domain. The protein may be used in any suitable format; in one non-limiting embodiment, the protein may be a surface-bound Fc fusion protein, or secreted Fe fusion protein.

In all of the above embodiments, the antibody detection reagents can be labeled with a detectable label. In one embodiment, the detectable labels for reagents to detect autoantibodies against one protein are distinguishable from the detectable labels to detect autoantibodies against the other protein. Methods for detecting the label include, but are not limited to spectroscopic, photochemical, biochemical, immunochemical, physical or chemical techniques. Any suitable detectable label can be used.

The compositions can be stored frozen, in lyophilized form, or as a solution. In one embodiment, the compositions can be placed on a surface of a solid support. Any suitable solid support may be used. Examples of such supports include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethacrylate polymers; sol gels; porous polymer hydrogels; nanostructured surfaces; nanotubes (such as carbon nanotubes), and nanoparticles (such as gold nanoparticles or quantum dots). This embodiment facilitates use of the compositions in various detection assays. For example, anti-IgG can be used to precoat the wells of a microwell plate and the antibody detection reagents (such as the proteins discussed herein) can be added to the precoated wells.

In a second aspect, the present invention provides methods for monitoring breast cancer therapy, comprising
(a) contacting a bodily fluid sample from a subject who is undergoing or has undergone breast cancer therapy with one or more reagents for detecting autoantibodies against one or more proteins selected from the group consisting of A1AT, ANGPTL4, LRP10, GFRA1, LGALS3, CST2, DKK1, CAPC, GRP78, and GRN; and
(b) determining an amount of autoantibodies against the one or more proteins in the bodily fluid sample;
wherein a decrease in the amount of autoantibodies relative to a baseline level of autoantibodies in a control, such as a similar bodily fluid sample from the subject indicates efficacy of the breast cancer therapy in the subject.

In a further aspect, the invention provides methods for prognosing breast cancer recurrence, comprising
(a) contacting a bodily fluid sample from a subject who has received breast cancer therapy with one or more reagents for detecting autoantibodies against one or more proteins selected from the group consisting of A1AT, ANGPTL4, LRP10, GFRA1, LGALS3, CST2, DKK1, CAPC, GRP78, and GRN; and
(b) determining an amount of autoantibodies against the one or more proteins in the bodily fluid sample;
wherein an increase in the amount of autoantibodies relative to a baseline level of autoantibodies in a control, such as a similar bodily fluid sample from the subject indicates a likelihood of breast cancer recurrence in the subject.

As disclosed in the examples that follow, a decrease in the amount of autoantibodies of the one or more recited markers over time compared to a baseline level (i.e.: before breast cancer therapy initiation) indicates a favorable treatment response, while no decrease, or an increase, in autoantibody levels indicates a non-favorable treatment response. The methods can be carried out at any suitable time after breast cancer therapy begins as determined by attending medical personnel in light of all factors. In various non-limiting embodiments, the methods may be carried out at least 1 week, 2 weeks, 3 weeks, 1 month, 2 months, 3 months, 4 months, 5 months, 6 months, 7 months, 8 months, 9 months, 10 months, 11 months, 12 months, 18 months, 24 Months, etc. after the beginning of therapy. As will be understood by those of skill in the art, the methods can be carried out any number of times for a given subject as deemed appropriate by attending medical personnel. Thus, the methods can be carried out 1, 2, 3, 4, 5, 6, 7 8, 9, 10, or more times for a given subject, to monitor the course of therapy. In one non-limiting embodiment, the methods are carried out at 6 months and/or 12 months after initiation of breast cancer therapy. As will be understood by those of skill in the art, the methods can be carried out during the therapy, and can also be carried out after completion of the therapy, to monitor for possible breast cancer recurrence.

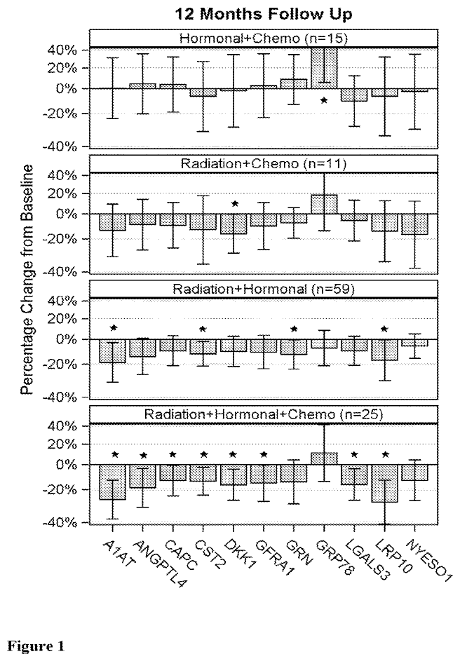

The subject may be any suitable subject receiving breast cancer therapy, including but not limited to a human subject. As used herein, “breast cancer therapy” includes one or more of surgery to remove the primary breast tumor, radiation therapy, chemotherapy, HERCEPTIN® therapy, and hormonal therapy. In one non-limiting embodiment, the subject has had surgery to remove the primary breast tumor and is receiving additional therapy selected from radiation therapy, chemotherapy, HERCEPTIN® therapy and/or hormonal therapy. Significant decreases in levels of response between baseline and 12 month time points against 9 TAAs (i.e. A1AT, ANGPTL4, CAPC. CST2, DKK1, GFRA1, GRN, LGALS3 and LRP10) were observed in three treatment groups. Radiation+chemotherapy, radiation+hormonal therapy, and radiation+hormonal therapy+chemotherapy had average geometric mean decreases for the 9 significant TAA of −11%, −13%, and −18%, respectively (FIG. 1). Autoantibodies against DKK1 significantly decreased in the radiation+chemotherapy group. The radiation+hormonal therapy group contained significant decreases in autoantibody response against A1AT, CST2, GRN and LRP10. In the Radiation+Hormonal therapy+chemotherapy group, A1AT and LRP10 had the greatest decrease in autoantibody response levels (−26% and −28%, respectively) compared to the other antigens and regimens. The triple therapy of radiation+hormonal+chemotherapy was more effective at reducing the autoantibody responses against the TAAs than any other combination of treatment. This group had significant decreases in autoantibody responses against all 9 TAAs.

In one embodiment, the one or more reagents comprise 2, 3, 4, or all 5 proteins, or antigenic fragments thereof, selected from the group consisting of ANGPTL4, GFRA1, LGALS3, DKK1 and GRN.

The antibody detection markers may be any suitable reagents that can be used to detect antibodies against the recited proteins, including but not limited to the recited protein, a secreted version of the protein (such as a native secreted form of the protein), or an extracellular domain of the protein. Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular protein. Thus, in one embodiment the antibody detection marker can be a secreted or membrane portion of the recited protein. Exemplary amino acid sequences of the secreted or membrane portion of the recited proteins are as disclosed herein. Thus, the reagents for use can be any suitable one or more reagents for detecting autoantibodies against one or more proteins selected from the group consisting of A1AT, ANGPTL4, LRP10, GFRA1, LGALS3, CST2, DKK1, CAPC, GRP78, and GRN, secreted portions thereof, or membrane portions thereof, including but not limited to the reagents of any embodiment of the compositions of the invention, or combinations thereof. In various non-limiting embodiments, the reagents comprise reagents for detecting human autoantibodies against at least three, four, five, six, seven, eight, or all nine proteins in the recited group. In another non-limiting embodiment, the reagents comprise the recited proteins or antigenic fragments thereof. Such proteins may be in a native form. In another embodiment, the reagents comprise at least two proteins, or antigenic fragments thereof, and wherein the at least two proteins, or antigenic fragments thereof, are expressed as a fusion with a detectable domain. The protein may be used in any suitable format; in one non-limiting embodiment, the protein(s) may be a surface-bound Fe fusion protein or secreted Fe fusion protein. In another embodiment, the reagents can be delectably labeled. In another embodiment, the reagents may be bound to a surface of a solid support.

In various embodiments, at least 1, 2, 3, 4, or 5 of the antibody detection markers are selected from the group consisting of A1AT, GFRA1, LGALS3, CAPC, and CST2. In various further embodiments, at least 1, 2, 3, 4, or 5 of the antibody detection markers are selected from the group consisting of ANGPTL4, GFRA1, LGALS3, DKK1 and GRN

As shown in the examples that follow, GRP78 was the only TAA to exhibit a significantly increase autoantibody response, which occurred in the hormonal therapy+chemotherapy group. Thus, in another aspect, the invention provides methods for monitoring breast cancer therapy, comprising
(a) contacting a bodily fluid sample from a subject who is undergoing or has undergone breast cancer therapy with one or more reagents for detecting autoantibodies against GRP78; and
(b) determining an amount of autoantibodies against GRP78 in die bodily fluid sample;
wherein an increase in the amount of autoantibodies against GRP78 relative to a control, such as a baseline level of autoantibodies in a similar bodily fluid sample from the subject indicates efficacy of the breast cancer therapy in the subject.

In another aspect, the invention provides methods for prognosing breast cancer recurrence, comprising
(a) contacting a bodily fluid sample from a subject who has received breast cancer therapy with one or more reagents for detecting autoantibodies against GRP78; and
(b) determining an amount of autoantibodies against GRP78 in the bodily fluid sample;
wherein a decrease in the amount of autoantibodies relative to a baseline level of autoantibodies against GRP78 in a control, such as a similar bodily fluid sample from the subject indicates a likelihood of breast cancer recurrence in the subject.

In one embodiment of each of these aspects, the subject is receiving or has received hormonal therapy and chemotherapy.

As will be understood by those of skill in the art, the methods may include the use of additional antibody detection markers and controls as is appropriate for an intended use of the composition. The contacting can be carried out under any suitable conditions for promoting binding between the autoantibodies in the bodily fluid sample and the reagent to forma binding complex that can be detected. Appropriate such conditions can be determined by those of skill in the art based on the intended assay, in light of the teachings herein. Similarly, any suitable additional steps can be used in the methods, such as one or more wash or other steps to remove unbound reagents.

Any suitable detection technique can be used, including but not limited to enzyme linked immunosorbent assays (ELISA), bead based assay platforms such as the LUMINEX® systems, 2-D array based assay platforms such as SEARCHLIGHT®, and the INANOVATE® ‘Longitudinal Assay Screening’ platform which may be capable of quantitating all the listed breast cancer biomarker from patient samples at their clinically relevant concentrations in a single test and dilution. In one embodiment, the compositions can be placed on a solid support, such as in a microarray, glass slide, membrane, microplate format or beads. The embodiment facilitates use of the compositions. Exemplary such assays are provided in the examples.

Similarly, any suitable bodily fluid can be used, including but not limited to a serum sample, plasma sample or blood sample from the subject. A “plasma sample” means blood plasma, the liquid component of blood, and is prepared, for example, by centrifugation of whole blood to remove blood cells. A serum sample is a plasma sample in which blood clotting factors have been removed.

In one embodiment, when no decrease is determined in the amount of autoantibodies relative to a baseline level of autoantibodies in a similar bodily fluid sample from the subject, the method further comprises altering the breast cancer therapy being administered to the subject. Since the lack of autoantibody decrease indicates a non-favorable therapeutic outcome for the subject, this embodiment permits modifying the therapy as deemed appropriate by attending medical personnel (i.e.: increased dosage, change in treatment, etc.) to achieve a more favorable therapeutic outcome.

In another embodiment, the methods of any embodiment or combination of embodiments are used to detect breast cancer recurrence. When no decrease (or an increase) is determined in the amount of autoantibodies relative to a baseline level of autoantibodies in a similar bodily fluid sample from the subject, the methods provide an indication of breast cancer recurrence. The fact that the autoantibody levels of patients decrease over the course of treatment supports the potential of using the detection of these autoantibody levels as a prognostic indication of recurrence. If the cancer recurs, the autoantibody levels increase and this increase would be detected by the assay.

### Example 1: Longitudinal Autoantibody Responses Against Tumor-Associated Antigens Decrease in Breast Cancer Patients According to Treatment Modality

Early diagnosis of breast cancer (BCa) is critical for increased disease survival, both at the time of initial disease as well as for recurrence (1, 2). The 5-year survival rate for women diagnosed with local BCa is 98.6%. Survival declines to 83.8% for regional stage and plummets to 23.3% for distant stage (3). Only 5% of U.S. women present with metastatic BCa when first diagnosed (4); however, incurable metastatic disease is responsible for most BCa-related deaths (5). Unfortunately, recurrence of BCa is most often found when patients report symptoms, such as shortness of breath, chronic cough, headache, weight loss or bone pain. Once a patient is diagnosed with metastatic BCa, the intent for treatment is no longer curative; instead, the goal is to control the disease for as long as possible (6, 7). Presently, no alternative screening methods are recommended for asymptomatic patients without clinical findings on physical examination (8). By the time the metastases are identified by physical symptoms, the patient's chances of disease-free survival are greatly diminished. Lack of early detection of recurrence for asymptomatic patients is a factor in the inability to cure metastatic BCa.

Early detection of the primary breast cancer (BCa) tumor allows physicians to treat a patient with the intent to cure. However, most BCa-related deaths are due to incurable metastatic disease and not the primary tumor. It is currently unknown what changes occur to patient autoantibody levels after primary diagnosis of BCa and treatment. A LUN′HNEX® multiplex bead assay was developed to allow simultaneous measurement of autoantibody responses against 32 conformation-carrying TAAs in a single patient sample. The antigens were selected from a membrane-associated polyribosomal cDNA library (MAPcL), which encodes membrane and secreted proteins highly expressed in BCa and should preferentially induce an antibody response in patients. The conformation of membrane and secreted proteins is particularly important because discontinuous epitopes will only be present for antibody recognition when the antigen is folded properly. Expression constructs were generated to encode the extracellular portion of the TAA fused to rabbit Fc (rFc). A eukaryotic expression system was developed to produce conformation-carrying antigens that are processed through the endoplasmic reticulum and Golgi to generate antigens with post-translational modifications.

LUMINEX® bead-based multiplex technology allows measurement of the interaction of patient autoantibodies with a panel of antigen biomarkers enabling quantitation across all biomarkers in a single test. The LUMINEX® xMAP® microsphere technology (Luminex, Austin, Tex.) is based on color-coded, 5.6-micron beads called microspheres. These beads are internally labeled with two different fluorescent dyes with different levels for each region, which are identified in the mixture by different red/infrared emission spectra. Simultaneously, the LUMINEX® instrument measures the amount of the autoantibody captured by the TAAs preloaded on the beads as the fluorescent level of the secondary antibody in a separate detector channel. The LUMINEX® beads have a much smaller surface area on which to immobilize the capture antibody as compared to the area of a microplate well; therefore, smaller patient sample volumes were required and non-specific binding to the plastic surfaces was reduced.

Three serial blood draws were collected from 200 BCa patients, before treatment, 6 and 12 months after surgery. These samples were assayed for autoantibody responses against 32 TAAs, and the levels present in the follow up samples were compared to autoantibody levels at the time of surgery. Patients were categorized according to treatment regimen, including surgery, chemotherapy, radiation, trastuzumab and hormonal therapies. Autoantibody responses against 9 TAAs (A1AT, ANGPTL4, CAPC, CST2, DKK1, GFRA1, GRN, LGALS3 and LRP10) were significantly reduced at 12 months after surgery with treatment regimens including radiation chemotherapy, radiation+hormonal therapy, or radiation+hormonal+chemotherapy. Autoantibody levels were generally lower at 12 months than 6 months after surgery, consistent with the patient's tumor burden decreasing over the course of treatment. Consequently, this data indicates that autoantibody levels increase in response to metastasis, and detection of this change can be used as an early indicator of recurrence.

### Materials and Methods

### Plasmid Construction and Protein Production

Briefly, the extracellular domain of transmembrane proteins or the full-length sequence of secreted and intracellular proteins was cloned into pSecTag2-rabbit Fe or pFUSE-rFc1. These plasmids included a secretion signal, as well as a C-terminal rabbit Fc (rFc) tag. The TAA-rFc plasmids were transfected into 293T cells with EFFECTENE™ (Qiagen, Valencia, Calif.), and the encoded proteins were secreted into the cell culture supernatant. Supernatants were harvested after transfection, and TAA-rFc content was measured with an anti-rFc sandwich ELISA.

### Antibody Coupling to Magnetic Beads

Goat anti-rabbit IgG Fc antibody (Jackson Immunoresearch, West Grove, Pa.) was coupled to LUMINEX® beads utilizing the xMAP® AbC Kit (Luminex, Austin, Tex.) according to the instruction of the manufacturer. In brief, beads were activated with EDC (1-Ethyl-3-[3-dimethylaminopropyl]carbodiimide hydrochloride) and Sulfo-NHS (N-hydroxysulfosuccinimide) for 20 minutes. After washing with phosphate buffered saline (PBS, pH 7.4), anti-rFc antibody was added to the beads at a concentration of 20 μg per 1×10⁶ beads and incubated for two hours shielded from light. The beads were washed again and stored at 4° C. shielded from light until use. Coupling was performed on 32 LUMINEX® bead regions that can be mixed for use and distinguished in 32 different areas by a LUMINEX® 100/200 instrument (Millipore).

### Loading of Tumor-Associated Antigen-Rabbit Fc Fusion Proteins to Beads

Beads coupled with the anti-rFc antibody were coated with each TAA-rFc fusion protein by incubation with the cell culture supernatant containing secreted TAA-rFc fusion protein. Each of the 32 TAA-rFe fusions was bound to one LUMINEX® bead region. Beads were incubated with the fusion protein at 40 μg 10⁶ beads overnight at 4° C. Beads were stored in PBS-TBN buffer (PBS with 0.1% bovine serum albumin, 0.02% Tween 20 and 0.05% sodium azide) at 4° C. in the dark until use.

### Patients

The inclusion criteria for cases were women over 30 years of age that were newly diagnosed with BCa (any type) at Sanford Health, Sioux Falls, S. Dak. Patients provided one 10 ml EDTA tube of blood prior to mastectomy, lumpectomy, radiation therapy, chemotherapy or other treatment. Potential subjects were excluded if they had a previous history of cancer of any kind. Blood samples from 200 BCa patients were collected from Oct. 8, 2009 to Apr. 17, 2012. Patients enrolled in the study were followed for one year after surgery, and blood draws were obtained at follow-up oncology visits at 6 and 12 months after surgery. Follow-up draws were collected from Apr. 15, 2010 to Aug. 27, 2013. All patients provided written informed consent, and the Sanford Health IRB approved the study protocol.

### Plasma Collection and Storage

The 10 ml EDTA tube was centrifuged at 2000×g for 10 minutes within 12 hrs of drawing. Plasma was collected as the supernatant, placed in aliquots and stored at −80° C. until the assay for the autoantibodies.

### Multiplex Autoantibody Bead Assay

Autoantibodies against the 32 TAAs in the plasma samples were measured simultaneously in a single well utilizing a multiplex bead assay. The xMAP® LUMINEX® magnetic beads from 32 distinct regions, each coated with a different TAA-rFc fusion, were combined and distributed to the wells of a 96-well round bottom plate. Plasma samples from BCa patients were diluted 1:10 in FACS buffer (PBS with 5% fetal bovine serum and 0.1% sodium azide), and 200 μl of diluted plasma was applied to the beads in a single well. Samples were incubated with beads for 2 hours on ice. Beads were then pelleted magnetically and washed twice, with a final aspiration leaving only the beads. As the secondary antibody, R-Phycoerythrin (PE)-labeled goat anti-human IgG (Jackson Immunoresearch, West Grove, Pa.) was diluted 1:200 in FACS buffer and 200 μl was added to the beads of each well. After an incubation of one hour on ice, beads were washed twice. The beads in each well were re-suspended in 200 μl of FACS buffer and analyzed on a LUMINEX® 100/200 instrument, with a minimum of 100 events analyzed for each bead region. Each plate included a secondary only negative control, as well as a PE goat anti-rabbit IgG (Jackson ImmunoResearch, West Grove, Pa.) reacting with bead-loaded TAA-rFc fusions as a positive control. All washing and aspiration steps were performed with a Biotek ELx405 microplate washer with magnetic capabilities.

### Statistical Methods

Each 96-well plate had a negative control background and a positive control standard for all autoantibodies. In addition, each patient had her baseline. 6 month and 12 month samples analyzed in the same 96-well plate. For each autoantibody, the patient's median fluorescent intensity (WI) value had its plate MFI background level subtracted and was then shifted by the minimum constant to make all values at least one. The value was then normalized by the ratio of the MFI for the standard over the mean standard MFI across all 8 plates. Lastly the values were log transformed to stabilize the variance and produce a more symmetric distribution, i.e. autoantibody response=LN[(autoantibody−background+constant)*(standard−background)/mean(standard−background)]. To measure precision, the inter-assay CV for each TAA was calculated across the plates for the positive and negative controls.

A repeated measure ANOVA was used to model the geometric mean changes from baseline for each autoantibody over time (i.e. 6 and 12 months) as an exploratory analysis, with a compound symmetry correlation structure. The models included all interactions among indicator variables for radiation, hormonal and chemotherapy with time. This approach included 183 out of 200 BCa patients in the primary analysis; the 17 patients given trastuzumab (HERCEPTIN® as part of their treatment consisted of 4 groups and were analyzed secondarily by examining their mean response profile. Point estimates and 95% confidence intervals (CI) were calculated for all 8 treatment combinations, which did not include HERCEPTIN®, at 6 and 12 months in order to rank autoantibodies by the number of positive findings. Since each TAA had 16 (8 treatments at 6 and 12 months) point estimates tested, one false positive was expected for each TAA; therefore, the observed geometric mean changes were presented for autoantibody levels with 3 or more positive findings. A p-value<0.05 was used to ascribe statistical significance, and SAS (Cary, N.C.) version 9.3 was used for all analyses.

### Results

Serial plasma samples were collected from 200 newly diagnosed BCa patients (13). Characteristics of the 200 BCa patients enrolled in this study have been previously described by our laboratory, including demographic information, tumor size, tumor marker status, in site versus invasive components and lymph node involvement (13). Blood draws were acquired before treatment, 6 months and 12 months after surgical resection of the primary tumor. To determine the patients' autoantibody responses against cancer antigens over the course of treatment, 32 TAA-rFc fusion proteins consisting of 20 previously analyzed TAAs (13) and 12 newly selected cancer antigens (Table 1) were generated. The eukaryotic expression system developed previously (13) was used to generate all of the TAA-rFc conformation-carrying antigens for the multiplex immunoassay. Thirty-two sets of LUMINEX® beads consisting of unique red/infared emission spectra were coated with anti-rabbit IgG. The 32 TAA-rFc fusion proteins were attached to the coated LUMINEX® beads followed by incubation with plasma samples acquired from patients before treatment, 6 months and 12 months post-surgery. The average inter-assay CV for the 32 autoantibody responses measured at baseline (before the start of treatment) with the LUMINEX® multiplex bead platform were 11.1% and 11.4% for the low and high controls, respectively (Table 2). In comparison, the average inter-assay CV for the blocking buffer control of the ELISA platform autoantibody assay was 12.4% (13). The LUMINEX® multiplex system greatly increased assay throughput and slightly improved the reproducibility.

TABLE 1
Additional candidates for generation of rFc fusion proteins

| | | Signal Sequence | Encoded Amino |
|---|---|---|---|
| Gene | Accession # | Amino Acids | Acid Fragment |
| A1AT | NM_000295.4 | 1-24 | 25-418 |
| AMACR | NM_014324.5 | None | 1-382 |
| BIRC5 | NM_001168.2 | None | 1-142 |
| CALD1 | NM_033139.3 | None | 1-558 |
| CAPC | NM_001013653.2 | 1-26 | 27-264 |
| CCNB1 | NM_031966.3 | None | 1-433 |
| CCND1 | NM_053056.2 | None | 1-295 |
| GRP78 | NM_005347.4 | None | 1-654 |
| LGALS3 | NM_002306.3 | None | 1-250 |
| MYC | NM_002467.4 | None | 1-439 |
| NY-ESO-1 | NM_001327.2 | None | 1-180 |
| XAGE1 | NM_001097594.2 | None | 1-81 |

TABLE 2
Inter-assay coefficient of variation for negative and
positive controls for 32 TAA on the bead platform

| | | |
|---|---|---|
| | Negative Control | Positive Control |
| Autoantibody | (Background) | (Standard) |

Original 20

| | | |
|---|---|---|
| ANGPTL4 | 9.2 | 12.3 |
| CD147 | 5.7 | 10.7 |
| CD320 | 21.8 | 12.9 |
| CDH3 | 7.5 | 12.3 |
| CST2 | 13.4 | 12.2 |
| DKK1 | 6.6 | 9.7 |
| EPHA2 | 5.2 | 12.6 |
| GFRA1 | 9.6 | 11.5 |
| GRN | 11.7 | 15.4 |
| IGFBP2 | 14.7 | 9.7 |
| LAMC2 | 7.7 | 11.1 |
| LRP10 | 5.5 | 10.3 |
| LRRC15 | 5.7 | 16.1 |
| MUC1 | 8.3 | 10.1 |
| SPINT2 | 12.9 | 9.2 |
| SPON2 | 14.3 | 10.5 |
| SSR2 | 18.2 | 11.2 |
| SUSD2 | 7.0 | 12.3 |
| LGALS1 | 14.7 | 11.7 |
| ERBB2 | 9.3 | 12.2 |

Additional 12

| | | |
|---|---|---|
| A1AT | 12.2 | 12.8 |
| AMACR | 11.3 | 11.2 |
| BIRC5 | 12.8 | 11.3 |
| CALD1 | 11.8 | 9.9 |
| CAPC | 10.2 | 10.4 |
| CCNB1 | 12.8 | 9.2 |
| CCND1 | 10.9 | 10.3 |
| GRP78 | 11.9 | 11.7 |
| LGALS3 | 13.1 | 13.9 |
| MYC | 12.5 | 9.5 |
| NY-ESO-1 | 15.9 | 11.2 |
| XAGE1 | 11.3 | 10.9 |
| Average | 11.1 | 11.4 |

Each subject was categorized based on the treatments received during the 12 months following initial diagnosis. All enrolled BCa patients in our study underwent surgery to remove the primary tumor, including a breast lumpectomy or mastectomy. In addition to surgery, treatment included combinations of hormonal therapies, HERCEPTIN®, radiation and cytotoxic chemotherapy (Table 3). Seventy-two patients received cytotoxic chemotherapy consisting of one of the following regimens: adriamycin/cytoxan+taxol=33 patients; cytoan+taxol=22 patients; adriamycin/cytoxan=5 patients; carboplatin+taxol 6 patients; adriamycin+cytoxan/taxol=3 patients; adriamycin/cytoxan carboplatin/Gemzar=1 patient; carboplatin+taxotere+novantrone=1 patient; taxol alone=1 patient. Patients were not analyzed according to the subclass of cytotoxic chemotherapy drugs used for treatment. If a patient had any one of the chemotherapy regiments described above, they were considered part of the chemotherapy treatment group. Grouping the study participants based on treatment regimen resulted in 12 groups, ranging from no treatment after surgery to all treatment modalities administered (Table 3). Eight of the twelve groups received a combination of at least two therapies in addition to surgery. Three groups received a single therapy in addition to surgery, and one group received surgery alone.

TABLE 3
Number of Patient Blood Draws Per Treatment Modality and Visit

| Treatment Group* | Baseline | | 6 Month | 12 Month | Subtotal |
|---|---|---|---|---|---|
| Radiation + Hormonal | 59 | (29.5%) | 52 | 52 | 163 |
| Hormonal | 31 | (15.5%) | 25 | 26 | 82 |
| Radiation + Hormonal + Chemotherapy | 25 | (12.5%) | 23 | 22 | 70 |
| Surgery Only | 24 | (12.0%) | 15 | 10 | 49 |
| Hormonal + Chemotherapy | 15 | (7.5%) | 12 | 13 | 40 |
| Radiation | 13 | (6.5%) | 8 | 8 | 29 |
| Radiation + Chemotherapy | 11 | (5.5%) | 11 | 11 | 33 |
| Radiation + Hormonal + Chemotherapy + | 8 | (4.0%) | 8 | 8 | 24 |
| HERCEPTIN ® | | | | | |
| Chemotherapy | 5 | (2.5%) | 4 | 5 | 14 |
| Hormonal + Chemotherapy + HERCEPTIN ® | 5 | (2.5%) | 5 | 5 | 15 |
| Radiation + Chemotherapy + HERCEPTIN ® | 3 | (1.5%) | 2 | 2 | 7 |
| Radiation + Hormonal + HERCEPTIN ® | 1 | (0.5%) | 1 | 1 | 3 |
| Total number of samples | 200 | (100%) | 166 | 163 | 529 |

*All patients received surgery to remove the primary tumor

The primary exploratory analysis included 183 out of 200 patients and encompassed the 8 patient treatment groups not receiving HERCEPTIN® therapy (Table 3). A repeated measure ANOVA was used to model the geometric mean changes from baseline for each autoantibody at 6 and 12 months, and the model included all interactions among indicator variables for radiation, hormonal therapy and chemotherapy with time. If the ANOVA model predicted at least 3 significant changes for an antigen among the 16 estimates (8 groups*2 time points consisting of 6 and 12 month blood draws), it was chosen for further analysis. Using this variable selection criterion, the model identified 11 antigens that were likely to have significantly modulated autoantibody signals in response to treatment in the 12 months following surgery. The TAAs chosen for further study included: A1AT, ANGPTL4, CAPC, CST2, DKK1, GFRA1, GRN, GRP78, LGALS3, LRP10 and NY-ESO-1.

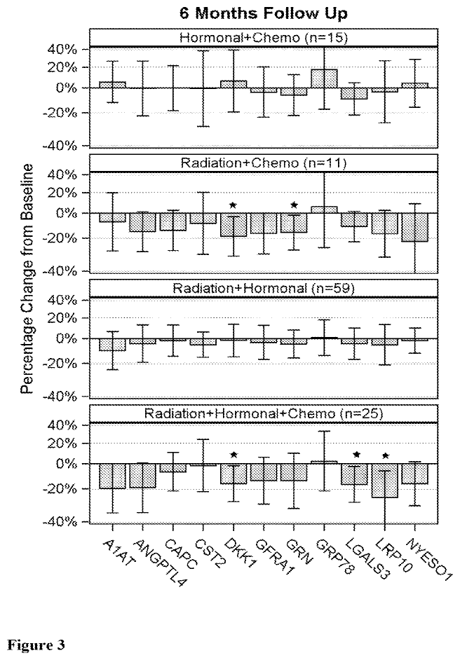

The actual geometric mean changes from baseline were calculated for the 11 TAAs at 6 and 12 months (FIGS. 1 and 3, respectively). No significant changes in the autoantibody response against the 11 antigens were observed for patients who received surgery alone or were treated with surgery and a single monotherapy: hormonal therapy, chemotherapy or radiation. At the 12-month time point, GRP7S was the only TAA to exhibit a significant increase autoantibody response of 44%, which occurred in the hormonal therapy+chemotherapy group (FIG. 1). While NY-ESO-1 was predicted to have significant changes in levels of response in the repeated measures ANOVA model, the observed changes were not significant (FIG. 1). However, significant decreases in levels of response between baseline and 12 month time points against 9 of the 11 TAAs (i.e. A1AT, ANGPTL4, CAPC, CST2, DKK1, GFRA1, GRN, LGALS3 and LRP10) were observed in three treatment groups. Radiation+chemotherapy, radiation+hormonal therapy, and radiation+hormonal therapy+chemotherapy had average geometric mean decreases for the 9 significant TAA of −11%, −13%, and −18%, respectively (FIG. 1). In the Radiation+Hormonal therapy+chemotherapy group, A1AT and LRP10 had the greatest decrease in autoantibody response levels (−26% and −28%, respectively) compared to the other antigens and regimens. The triple therapy of radiation+hormonal+chemotherapy was more effective at reducing the autoantibody responses against the TAAs than any other combination pair of treatment. In addition, the decrease in response of the geometric mean was −15% at 6 months after surgery (FIG. 3) compared to a greater decrease of −18% at the 12 month time point. It is apparent that autoantibody levels diminish during the course of treatment, and they continue to decrease with time as the patient is further removed from initial diagnosis.

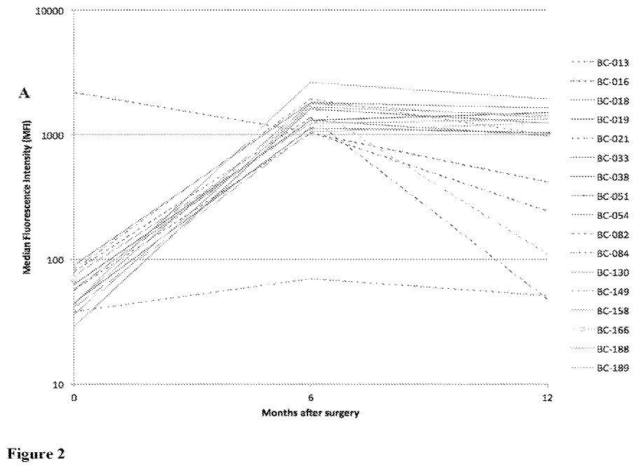

Four of the 12 treatment groups (totaling 17 patients) were treated with HERCEPTIN® therapy as follows: radiation+hormonal+chemotherapy+HERCEPTIN®=8 patients; hormonal+chemotherapy+HERCEPTIN®=5 patients; radiation+chemotherapy+HERCEPTIN®=3 patients; radiation+hormonal HERCEPTIN®=1 patient (Table 2). HERCEPTIN® antibody therapy is generally administered every three weeks for one year (27). We confirmed that the HERCEPTIN® antibody circulating in the patient's blood was generating a signal against the ERBB2 antigen using the LUMINEX® bead based assay. The geometric mean levels in the ERBB2 antibody response increased from 48 median fluorescence intensity (MFI) at baseline to 1206 MFI at 6 months, and then decreased to 600 MI′ at 12 months (FIG. 2). The changes compared to baseline were significant at 6 and 12-months (both p<0.0001), but there was no significant difference between 6 and 12-month levels (p=0.09). Most patients had a high response against ERBB2 at the 6-month time point, which is consistent with the fact that patients were receiving HERCEPTIN® therapy spanning this time period. Patient BC-166 (FIG. 2, light green circle) received her treatments at an off-site medical center, and therefore, the 6 and 12-month blood draws were not acquired. Six patients (FIG. 2, dashed lines) discontinued HERCEPTIN® therapy prior to their 12-month visit. Subject BC-021 had very low antibody response against ERBB2 which is consistent With her only receiving two treatments before discontinuing therapy. For the other five patients that had discontinued HERCEPTIN® therapy, their anti-ERBB2 antibody responses for the 12-month blood draws were inversely related to the number of months from the patients' last HERCEPTIN® infusions. Patient BC-082 discontinued HERCEPTIN® use after her 6-month visit, and by her 12-month visit the MFI value had returned to baseline. Patients BC-149, BC-013 and BC-84 were 4 months removed from treatment and had a level of 110 MFI, 244 MFI and 994 MFI, respectively. Patient BC-016 had her final blood draw at 3 months after her last HERCEPTIN® infusion, and her response level was 418 MFI. BC-013 had a strong endogenous autoantibody response to ERBB2 prior to treatment with HERCEPTIN®, yielding a high signal at the baseline time point (FIG. 2). The remaining 10 patients (BC-018, BC-019, BC-033, BC-038, BC-051, BC-054, BC-130, BC-158, BC-188 and BC-189) were continuing to receive HERCEPTIN® during the longitudinal blood draws, and the anti-ERBB2 antibody response against the ERBB2 antigen plateaued between the 6 and 12-month visits (FIG. 2).

### Discussion

This study provides important data on longitudinal change of autoantibody responses occurring in BCa patients after surgery. We were able to assess many TAAs simultaneously with our unique multiplex bead assay. In addition, rich data on pathology and treatment information allows the findings of this study to be associated with the treatment regimens administered to each patient. Our results indicate that the combination of the selected 9 antigens shows promise in developing an autoantibody assay toward early detection of metastatic disease.

The data collected from the analysis of longitudinal blood draws in this study indicates that autoantibody response to TAAs decreases as a function of time after the primary tumor is surgically resected and therapy is initiated (FIG. 1). The absence of a large tumor mass likely explains the reduced production of autoantibodies to TAAs. With the majority of the cancer cells removed, the immune system is exposed to fewer cancer antigens. Without continued exposure to the cancer antigens, the immune system reduces the levels of autoantibody production. Surprisingly, we also found that treatments administered after the removal of the primary tumor had a profound effect on the autoantibody profile of these patients. Significant changes in antibody responses can be detected at the 6-month time point (Table 2), With a more extensive reduction detectable 12 months after surgery (Table 3). The changes seen in these samples are attributed to the therapies administered to that patient, but it is acknowledged that these therapies are a function of the characteristics of each subtype of BCa.

To that end, the treatment modality groups that had the greatest decrease in autoantibody response levels were radiation+hormonal therapy; radiation+chemotherapy; and radiation+hormonal therapy+chemotherapy (FIG. 1). The common denominator of the three most affected groups for significant changes in autoantibody response levels is radiation treatment. However, radiation treatment alone is not enough to significantly decrease the response levels of the autoantibodies. Patients treated with a combination of all therapies (surgery, hormonal therapy, chemotherapy and radiation), had autoantibody response levels significantly decrease for 8 separate antigens (FIG. 1). Finally, the hormonal therapy+chemotherapy regimen contained a significant change in an autoantibody response against the GRP78 antigen. This group of patients did not receive radiation therapy, and the autoantibody response against GRP78 was unique because instead of decreasing, the response dramatically increased 12 months after surgery (FIG. 1).

The initial analysis of the data revealed that a group of patients had a high response against the ERBB2 antigen (FIG. 2). Upon further evaluation, we determined that the tumors removed from those patients were HER-2 amplified, and the patients were candidates for HERCEPTIN® therapy. HERCEPTIN® antibody recognizes and binds to a native pocket-like binding region of ERBB2 (33, 34). By using our conformation-carrying method of antigen generation, an ERBB2-rFc fusion protein was produced which mimicked the conformation of the native ERBB2 protein (13). Our assay was measuring the response of the HERCEPTIN® therapeutic antibody present in the peripheral circulation of the patient against the ERBB2 antigen attached to the beads (FIG. 2). Since HERCEPTIN® therapy is generally administered every three weeks by infusion for one year (27), this response served as an ‘in vivo human spiked control’ for our multiplex bead assay. HERCEPTIN® antibody present in the circulation of patients produced a high level of response against the ERBB2-rFc fusion protein corresponding to the timing of HERCEPTIN® treatments of patients. Previously, a phase IT study determined that the half-life of trastuzumab was 18 to 27 days when administered every 3 weeks (35). This length of time is consistent with the measured levels of the anti-ERBB2 antibody response over the course and end of trastuzumab treatment for the patients with HER-2 amplified tumors. For patients that had their final blood draw 3 and 4 months after finishing trastuzumab treatment, their antibody response against ERBB2 decreased dramatically. One patient had her blood drawn 6 months following her last trastuzumab infusion, and her response had returned to pretreatment levels (FIG. 2).

Of the 9 antigens reported in FIG. 2 whose autoantibody responses were significantly lowered over the course of treatment (A1AT, ANGPTL4, CAPC, CST2. DKK1, GFRA1, GRN, LGALS3 and LRP10), 4 were also present in the group of 7 antigens previously found to be predictive of the presence of BCa: ANGPTL4, DKK1, GERM and GRN (13).

### REFERENCES

1. Aragon R, Morgan J. Wong J H, Lum S. Potential impact of USPSTF recommendations on early diagnosis of breast cancer. Annals of surgical oncology. 2011; 18:3137-42.
2. Kontos M, Roy P, Rizos D, Petrou A, Hamed H, Contralateral relapse after surgery for breast cancer: evaluation of follow-up paradigms. International journal of clinical practice. 2013; 67:1113-7.
3. Siegel R, DeSantis C, Virgo K, Stein K, Mariotto A, Smith T, et al. Cancer treatment and survivorship statistics, 2012. CA Cancer J Clin. 2012; 62:220-41.
4. American Cancer Society. Breast Cancer Facts & Figures 2013-2014. [cited 2014 Mar. 27, 2014]; Available from: http://www.cancer.org
5. Lu J, Steeg P S, Price J E, Krishnamurthy S, Mani S A, Reuben J, et al. Breast cancer metastasis: challenges and opportunities. Cancer Res. 2009; 69:4951-3.
6. Kimbung S, Loman N, Hedenfalk I, Clinical and molecular complexity of breast cancer metastases. Seminars in cancer biology. 2015.
7. Cardoso F, Costa A, Norton L, Cameron D, Cufer T, Fallowfield L, et al. 1st International consensus guidelines for advanced breast cancer (ABC 1). Breast. 2012; 21:242-52.
8. Schneble E J, Graham L J, Shupe M P, Flynt F L, Banks K P, Kirkpatrick A D, et al. Current approaches and challenges in early detection of breast cancer recurrence. Journal of Cancer. 2014; 5:281-90.
9. Chapman C. Murray A, Chakrabarti J, Thorpe A. Woolston C, Sahin U, et al. Autoantibodies in breast cancer: their use as an aid to early diagnosis. Annals of oncology official journal of the European Society for Medical Oncology/ESMO. 2007; 18:868-71
10. Lacombe J, Mange A, Solassol J. Use of autoantibodies to detect the onset of breast cancer. Journal of immunology research. 2014; 2014:574981.
11. Henry N L, Hayes D F, Ramsey S D, Hortobagyi G N, Barlow W E, Gralow J R. Promoting quality and evidence-based care in early-stage breast cancer follow-up. Journal of the National Cancer Institute. 2014; 106:dju034.
12. Egland K A, Vincent J J, Strausberg R, Lee B, Pastan I. Discovery of the breast cancer gene BASE using a molecular approach to enrich for genes encoding membrane and secreted proteins. Proc Nati Aead Sci USA. 2003; 100:1099-104.
13. Evans R L, Pottala T V, Egland K A. Classifying Patients for Breast Cancer by Detection of Autoantibodies against a Panel of Conformation-Carrying Antigens. Cancer Prev Res 2014.
14. Carson R T, Vignali D A, Simultaneous quantitation of 15 cytokines using a multiplexed flow cytometric assay. J Immunol Methods. 1999; 227:41-52.
15. Egland K A, Liu X F, Squires S, Nagata S. Man Y G, Bera T K, et al. High expression of a cytokeratin-associated protein in many cancers. Proc Natl Acad Sci USA. 2006; 103:5929-34.
16. Fossa A, Alsoe L, Crameri R, Funderud S, Gaudernack G, Smeland E B, Serological cloning of cancer/testis antigens expressed in prostate cancer using cDNA phage surface display. Cancer immunology, immunotherapy: CII. 2004; 53:431-8.
17. Gonzalez-Gronow M, Cuchacovich M, Llanos C, Urzua C, Gawdi G, Pizzo S V. Prostate cancer cell proliferation in vitro is modulated by antibodies against glucose-regulated protein 78 isolated from patient serum. Cancer Res. 2006; 66:11424-31.
18. Liu W, De La Torre I G, Gutierrez-Rivera M C, Wang B, Liu Y, Dai L, et al. Detection of autoantibodies to multiple tumor-associated antigens (TAAs) in the immunodiagnosis of breast cancer. Tumour Biol. 2015; 36:1307-12.
19. Lopez-Arias E. Aguilar-Lemarroy A, Felipe Jave-Suarez L, Morgan-Villela G, Mariscal-Ramirez 1, Martinez-Velazquez M, et al. Alpha 1-antitrypsin: a novel tumor-associated antigen identified in patients with early-stage breast cancer. Electrophoresis. 2012; 33:2130-7,
20. O'Rourke D J, DiJohnson D A, Caiazzo R J, Jr., Nelson J C, Ure D, O'Leary M P, et al. Autoantibody signatures as biomarkers to distinguish prostate cancer from benign prostatic hyperplasia in patients with increased serum prostate specific antigen. Clinica chimica acta; international journal of clinical chemistry. 2012; 413:561-7.
21. Xie C, Kim H J, Haw J G, Kalbasi A, Gardner B K, Li G, et al. A novel multiplex assay, combining autoantibodies plus PSA has potential implications for classification of prostate cancer from non-malignant cases. Journal of translational medicine. 2011; 9:43.
22. Xu Y, Jin Y, Liu L, Zhang X, Chen Y, Wei J. Study of circulating IgG antibodies to peptide antigens derived from BIRCS and MYC in cervical cancer. FEBS open bio. 2015; 5:198-201.
23. Yang Z, Chevolot Y, Gehin T, Solassol J, Mange A, Souteyrand E, et al. Improvement of protein immobilization for the elaboration of tumor-associated antigen microarrays: application to the sensitive and specific detection of tumor markers from breast cancer sera. Biosensors & bioelectronics. 2013; 40:385-92.
24. Ye H, Sun C, Ren P, Dai L, Peng B, Wang K, et al. Mini-array of multiple tumor-associated antigens (TAAs) in the immunodiagnosis of breast cancer. Oncology letters. 2013; 5:663-8.
25. Zhou S L, Yue W B, Fan Z M, Du F, Liu B C, Li B, et al. Autoantibody detection to tumor-associated antigens of P53, IMP1, P16, cyclin B1, P62, C-myc, Survivn, and Koc for the screening of high-risk subjects and early detection of esophageal squamous cell carcinoma. Diseases of the esophagus: official journal of the International Society for Diseases of the Esophagus/ISDE. 2014; 27:790-7.
26. Zuo X, Chen L, Liu L, Zhang Z, Zhang X, Yu Q, et al. Identification of a panel of complex autoantigens (LGALS3, PHB2, MUC1, and GK2) in combination with CA15-3 for the diagnosis of early-stage breast cancer. Tumour Biol. 2015.
27. HERCEPTIN® Prescribing Information. Genentech, Inc. 2015.
28. Heo C K, Bahk Y Y, Cho E W, Tumor-associated autoantibodies as diagnostic and prognostic biomarkers. BMB reports. 2012; 45:677-85.
29. Liu W, Peng B, Lu Y, Xu W, Qian W, Zhang J Y. Autoantibodies to tumor-associated antigens as biomarkers in cancer immunodiagnosis. Autoimmun Rev. 2011; 10:331-5.
30. Zhu Q, Liu. M, Dai L, Ying X, Ye H, Zhou Y, et al. Using immunoproteomics to identify tumor-associated antigens (TAAs) as biomarkers in cancer immunodiagnosis. Autoimmun Rev. 2013; 12:1123-8.
31. Fernandez-Madrid F. Maroun M C, Autoantibodies in breast cancer. Advances in clinical chemistry. 2014; 64:221-40.
32. Lu H, Goodell V, Disis M L, Humoral Immunity Directed against Tumor Associated Antigens As Potential Biomarkers for the Early Diagnosis of Cancer. J Proteome Res. 2008; 7:1388-94.
33. Cho H S, Mason K, Ramyar K X, Stanley A M, Gabelli S B, Denney D W, Jr., et al. Structure of the extracellular region of HER2 alone and in complex with the Herceptin Fab. Nature. 2003; 421:756-60.
34. Garrett J T, Rawale S, Allen S D, Phillips G, Forni G, Morris J C, et al. Novel engineered trastuzumab conformational epitopes demonstrate in vitro and in vivo antitumor properties against HER-2/neu. J Immunol. 2007; 178:7120-31.
35. Leyland-Jones B, Gelmon K, Ayoub J P, Arnold A, Verna S, Dias R, et al. Pharmacokinetics, safety, and efficacy of trastuzumab administered every three weeks in combination with paclitaxel. J Clin Oncol. 2003; 21:3965-71.

We claim:

1. A composition comprising at least 2 antibody detection markers, wherein the antibody detection markers comprise reagents for detecting human autoantibodies against at least two proteins selected from the group consisting of A1AT (Alpha-1 antitrypsin) (SEQ ID NO:7), ANGPTL4 (Angiopoietin-like 4) (SEQ ID NO:1), LRP10 (LDL Receptor Related Protein 10) (SEQ ID NO:5), GFRA1 (GDNF Family Receptor Alpha 1) (SEQ ID NO:3), LGALS3 (Galectin-3) (SEQ ID NO:8), CST2 (Cystatin SA) (SEQ ID NO:6), DKK1 (Dickkopf WNT Signaling Pathway Inhibitor 1) (SEQ ID NO:2), CAPC (Cytokeratin-Associated Protein In Cancer) (SEQ ID NO:9), GRP78 (78 kDa glucose-regulated protein) (SEQ ID NO:12) and GRN (Granulin) (SEQ ID NO:4), wherein at least one of the proteins is selected from the group consisting of A1AT, LGALS3, and CAPC.

2. The composition of claim 1, wherein the composition includes reagents for detecting human autoantibodies against at least 3 proteins selected from the group consisting of A1AT, ANGPTL4, LRP10, GFRA1, LGALS3, CST2, DKK1, CAPC, GRP78, and GRN.

3. The composition of claim 1 or 2, wherein the composition includes reagents for detecting human autoantibodies against at least 5, 6, 7, 8, or all 9 proteins in the recited group.

4. The composition of any one of claims 1-3, wherein the composition consists of between 2 and 1000 antibody detection markers.

5. The composition of any one of claims 1-3, wherein the composition consists of between 4 and 500 antibody detection markers.

6. The composition of any one of claims 1-5, wherein the composition includes reagents for detecting human autoantibodies against at least 2 of ANGPTL4, GFRA1, LGALS3, DKK1 and GRN.

7. The composition of any one of claims 1-5, wherein the composition includes reagents for detecting human autoantibodies against at least 3, 4, or all 5 of ANGPTL4, GFRA1, LGALS3, DKK1 and GRN.

8. The composition of any one of claims 1-7, wherein the reagents for detecting human autoantibodies comprise the at least two proteins, or antigenic fragments thereof.

9. The composition of claim 8, wherein the at least two proteins or antigenic fragments thereof comprise native extracellular domains and/or native secreted proteins or antigenic fragments thereof.

10. The composition of any one of claims 1-9, wherein the reagents are detectably labeled.

11. The composition of any one of claims 8-10, wherein the at least two proteins, or antigenic fragments thereof, are expressed as a fusion with a detectable domain, including but not limited to an Fe domain.

12. The composition of any one of claims 1-11, wherein the reagents are immobilized on a surface of a solid support.

13. A method for monitoring breast cancer therapy, comprising
(a) contacting a bodily fluid sample from a subject who is undergoing or has undergone breast cancer therapy with one or more reagents for detecting autoantibodies against one or more proteins selected from the group consisting of A1AT, ANGPTL4, LRP10, GFRA1, LGALS3, CST2, DKK1, CAPC, GRP78, and GRN; and
(b) determining an amount of autoantibodies against the one or more proteins in the bodily fluid sample;
wherein a decrease in the amount of autoantibodies relative to a control, such as a baseline level of autoantibodies in a similar bodily fluid sample from the subject indicates efficacy of the breast cancer therapy in the subject.

14. A method for prognosing breast cancer recurrence, comprising
(a) contacting a bodily fluid sample from a subject who has received breast cancer therapy with one or more reagents for detecting autoantibodies against one or more proteins selected from the group consisting of A1AT, ANGPTL4, LRP10, GFRA1, LGALS3, CST2, DKK1 CAPC, GRP78, and GRN; and
(b) determining an amount of autoantibodies against the one or more proteins in the bodily fluid sample;
wherein an increase in the amount of autoantibodies relative to a baseline level of autoantibodies in a control, such as a similar bodily fluid sample from the subject indicates a likelihood of breast cancer recurrence in the subject.

15. The method of any one of claims 13-14, wherein the reagents comprise reagents for detecting autoantibodies 3 or more of the recited proteins.

16. The method of any one of claims 12-13, wherein the reagents comprise reagents for detecting autoantibodies against 5, 6, 7, 8, or all 9 of the recited proteins.

17. The method of any one of claims 13-16, wherein the one or more reagents comprise the composition of any one of claims 1-12.

18. The method of any one of claims 13-16, wherein the one or more reagents comprise at least 2 proteins, or antigenic fragments thereof, selected from the group consisting of ANGPTL4, GFRA1, LGALS3, DKK1 and GRN.

19. The method of any one of claims 13-16, wherein the one or more reagents comprise at least 3, 4, or all 5 proteins, or antigenic fragments thereof, selected from the group consisting of ANGPTL4, GFRA1, LGALS3, DKK1 and GRN.

20. A method for monitoring breast cancer therapy, comprising
(a) contacting a bodily fluid sample from a subject who is undergoing or has undergone breast cancer therapy with one or more reagents for detecting autoantibodies against GRP78 (SEQ ID NO:12); and
(b) determining an amount of autoantibodies against GRP78 in the bodily fluid sample;
wherein an increase in the amount of autoantibodies against GRP78 relative to a control, such as a baseline level of autoantibodies in a similar bodily fluid sample from the subject indicates efficacy of the breast cancer therapy in the subject.

21. A method for prognosing breast cancer recurrence, comprising
(a) contacting a bodily fluid sample from a subject who has received breast cancer therapy with one or more reagents for detecting autoantibodies against GRP78 (SEQ ID NO:12); and
(b) determining an amount of autoantibodies against GRP78 in the bodily fluid sample;
wherein a decrease in the amount of autoantibodies relative to a baseline level of autoantibodies against GRP78 in a control, such as a similar bodily fluid sample from the subject indicates a likelihood of breast cancer recurrence in the subject.

22. The method of any one of claims 13-21, wherein the contacting comprises use of ELISA.

23. The method of any one of claims 13-22, wherein the bodily fluid sample comprises a blood sample, a plasma sample, or a serum sample from the subject.

24. The method of any one of claims 13-23, wherein the subject has had surgery to remove the primary tumor.

25. The method of any one of claims 13-24, wherein the subject is receiving or has received radiation therapy and chemotherapy.

26. The method of any one of claims 13-25, wherein the contacting comprises use of Longitudinal Assay Screening, wherein all target biomarkers may be detected and quantitated within a single test and dilution.

27. The method of any one of claims 20-24 and 26, wherein the subject is receiving or has received hormonal therapy and chemotherapy.

28. The method of any one of claims 13-27, wherein no decrease is determined in the amount of autoantibodies relative to a baseline level of autoantibodies in a similar bodily fluid sample from the subject, and wherein the method further comprises altering the breast cancer therapy being administered to the subject.
